(19) United States
(12) Patent Application Publication
(10) Pub. No.: US 2008/0193990 A1
(43) Pub. Date: Aug. 14, 2008

(54) Tow-Step Method for Producing Polyesterols

(76) Inventors: Christoph Schnorpfeil, Dresden (DE); Mirko Kreitschmann, Mannheim (DE); Jean-Francois Stumbe, Strasbourg (FR); Gitta Egbers, Osnabruck (DE)

Correspondence Address: OBLON, SPIVAK, MCCLELLAND MAIER & NEUSTADT, P.C., 1940 DUKE STREET, ALEXANDRIA, VA, 22314, US

(21) Appl. No.: 11/909,108

(22) PCT Filed: Mar. 21, 2006
(86) PCT No.: PCT/EP2006/060898

§ 371 Date: Sep. 19, 2007

(30) Foreign Application Priority Data
Mar. 23, 2005 (DE) 102005014032.7

(51) Int. Cl. C12P 7/42; C08G 63/12

(52) U.S. Cl. 435/155; 528/288

(57) ABSTRACT

The present invention relates to a two-stage process for preparing polyesterols, which comprises the following process steps:
a) preparation of at least one base polyesterol by reaction of in each case at least one dicarboxylic acid with in each case at least one polyhydroxyl compound,
b) reaction of the base polyesterol from a) or a mixture of the base polyesterols from a) with at least one enzyme and, if appropriate, additionally with polyhydroxyl compounds.

The invention further relates to a polyesterol obtainable by the above process.

The present invention relates to a two-stage process for preparing polyesterols, which comprises the following process steps:
a) preparation of at least one base polyesterol by reaction of in each case at least one dicarboxylic acid with in each case at least one polyhydroxyl compound,
b) reaction of the base polyesterol from a) or a mixture of the base polyesterols from a) with at least one enzyme and, if appropriate, additionally with polyhydroxyl compounds.

Polymeric hydroxyl compounds such as polyesterols and polyetherols react with isocyanates to form polyurethanes which have a wide range of possible uses, depending on their specific mechanical properties. Polyesterols in particular are used for high-quality polyurethane products because of their favorable properties. The specific properties of the polyurethanes concerned depend strongly on the polyesterols used.

To produce polyurethanes, it is particularly important that the polyesterols used have a low acid number (cf. Ullmann's Encyclopedia, Electronic Release, Wiley-VCH-Verlag GmbH, Weinheim, 2000, under the keyword “Polyesters”, paragraph 2.3 “Quality Specifications and Testing”). The acid number should be very small since terminal acid groups react more slowly with diisocyanates than do terminal hydroxyl groups. Polyesterols having high acid numbers therefore lead to a lower buildup of the molecular weight during the reaction of polyesterols with isocyanates to form polyurethane.

A further problem associated with the use of polyesterols having high acid numbers for the polyurethane reaction is that the reaction of the numerous terminal acid groups with isocyanates forms an amide bond with liberation of carbon dioxide. The gaseous carbon dioxide can then lead to undesirable bubble formation. Furthermore, free carboxyl groups adversely affect the catalysis in the polyurethane reaction and also the stability of the polyurethanes produced toward hydrolysis.

On the basis of their chemical structure, polyesterols (also referred to as polyesters) can be divided into two groups, viz. the hydroxycarboxylic acid types (AB polyesters) and the dihydroxydicarboxylic acid types (AA-BB polyesters). The former are prepared from only one monomer by, for example, polycondensation of an ω-hydroxycarboxylic acid or by ring-opening polymerization of cyclic esters, known as lactones. On the other hand, AA-BB polyester types are prepared by polycondensation of two complementary monomers, generally by reaction of polyfunctional polyhydroxyl compounds (e.g. diols or polyols) with dicarboxylic acids (e.g. adipic acid or terephthalic acid).

The polycondensation of polyfunctional polyhydroxyl compounds and dicarboxylic acids to form polyesterols of the AA-BB type is generally carried out industrially at high temperatures of 160-280° C. The polycondensation reactions can be carried out either in the presence or absence of a solvent. However, a disadvantage of these polycondensations at high temperatures is that they proceed comparatively slowly. For this reason, esterification catalysts are frequently used to accelerate the polycondensation reaction at high temperatures. Classical esterification catalysts employed are preferably organic metal compounds, e.g. titanium tetrabutoxide, tin dioctoate or dibutyltin dilaurate, or acids such as sulfuric acid, p-toluenesulfonic acid or bases such as potassium hydroxide or sodium methoxide. These esterification catalysts are homogeneous and generally remain in the polyesterol after the reaction is complete. A disadvantage of this is that the esterification catalysts remaining in the polyesterol may adversely affect the later conversion of these polyesterols into the polyurethane.

A further disadvantage is the fact that by-products are frequently formed in the polycondensation reaction at high temperatures. Furthermore, the high-temperature polycondensations have to take place with exclusion of water in order to avoid the reverse reaction. This is generally achieved by the condensation being carried out under reduced pressure, under an inert gas atmosphere or in the presence of an entraining gas for the complete removal of the water.

Overall, the reaction conditions required, in particular the high reaction temperatures, the possible inert conditions or carrying out the reaction under reduced pressure and also the necessity of a catalyst lead to very high capital and operating costs for the high-temperature polycondensation.

To avoid these numerous disadvantages of the catalyzed condensation processes, alternative processes for preparing polyesterols in which enzymes are used at low temperatures in place of esterification catalysts at high temperatures have been developed. Enzymes used are generally lipases, including the lipases Candida antartica, Candida cylinderacea, Mucor meihei, Pseudomonas cepacia, Pseudomonas fluorescens.

In the known enzyme-catalyzed processes for preparing polyesterols of the AA-BB type, either “activated dicarboxylic acid components”, e.g. in the form of dicarboxylic acid diesters (cf. Wallace et al., J. Polym. Sci., Part A: Polym. Chem., 27 (1989), 3271) or “unactivated dicarboxylic acids” are used together with polyfunctional hydroxyl compounds. These enzymatic processes, too, can be carried out either in the presence or in the absence of a solvent.

Thus, for example, EP 0 670 906 B1 discloses a lipase-catalyzed process for preparing polyesterols of the AA-BB type at 10-90° C., which makes do without use of a solvent. In this process, it is possible to use either activated or unactivated dicarboxylic acid components.

Uyama et al., Polym. J., Vol. 32, No. 5, 440-443 (2000), also describe a process for preparing aliphatic polyesters from unactivated dicarboxylic acids and glycols (sebacic acid and 1,4-butanediol) in a solvent-free system with the aid of the lipase Candida antartica.

Binns et al., J. Polym. Sci., Part A: Polym. Chem., 36 2069-1080 (1998) disclose processes for preparing polyesterols from adipic acid and 1,4-butanediol with the aid of the immobilized form of the lipase B from Candida antartica (commercially available as Novozym 435®). In particular, the influence of the presence or absence of a solvent (in this case toluene) on the reaction mechanism was analyzed. It was able to be observed that the polyesterol is essentially extended only by stepwise condensation of further monomer units onto it in the absence of a solvent, while in the presence of toluene as solvent, transesterification reactions also play a role in addition to the stepwise formation of further ester links. Thus, the enzyme specificity of the lipase used appears to depend, inter alia, on the presence and type of the solvent.

However, the high-temperature polycondensations and the enzymatically catalyzed polycondensations for preparing polyesterols both have the disadvantage that the preparation of polyesterols by condensation reactions is carried out in plants for which a complicated periphery is necessary. In the case of the classical high-temperature polycondensation and also for the enzymatic polycondensation, facilities on the reactor for metering of liquids and/or solids are necessary. Water has to be removed from the reaction mixture under reduced pressure, by introduction of an inert gas or by means of an entrainer distillation. In addition, the water has to be separated off from the diols by distillation, since these have to remain in the reaction mixture as reaction partners for the acid component. Water and diols are generally separated using a distillation column. As an alternative, in the case of enzymatic processes it is also possible to use membranes which are permeable to water but not to the diols which are to be retained. Facilities for the generation of reduced pressure, e.g. pumps, for the separation of diols and water, e.g. distillation columns and membranes, or for the introduction of a stream of inert gas require high capital investment. In addition, particularly in the case of the high-temperature condensation, apparatuses for generating internal reactor temperatures of 160-270° C. are necessary.

The preparation of a very large and wide range of structurally different polyesterols can be carried out in many, small reactors. However, these small reactors all have to be provided with the complete periphery for the generation of reduced pressure, for the separation of diol/water mixtures and, if appropriate, for the generation of high temperatures. This requires an undesirably high specific capital investment. As an alternative, a large range of many, different polyesterols can also be prepared in a few, large reactors which require a small specific capital investment. However, the change between polyesterols of different composition and structure makes a cleaning step necessary on changing the product, which leads to a reduction in the utilization of capacity. In addition, the volume demand from customers can be smaller than the reactor volume for particular special products. In the preparation of such very small amounts, it is therefore unavoidable that the full reactor volume will not be utilized. This likewise leads to a decrease in capacity.

On the other hand, however, the preparation of a large range of structurally different special polyesterols having tailored properties (e.g. specific molecular weights, viscosities, acid numbers, etc.) is very desirable since these special polyesterols can in turn each be used for preparing specific polyurethanes which have properties in terms of molecular weight, functionality, glass transition temperature, viscosity, etc., which are tailored to their specific application.

It is therefore an object of the present invention to provide a process for preparing a very large range of special polyesterols having low acid numbers which avoids the disadvantages of the classical high-temperature processes and enzymatic processes for preparing polyesterols. In particular, such a process should be provided for preparing a large range of special polyesterols of the dihydroxydicarboxylic acid type having low acid numbers in which the high logistic and economic outlay required hitherto can be avoided.

This object is achieved according to the invention by a two-stage process for preparing polyesterols, which comprises the following process steps:
a) preparation of at least one base polyesterol by reaction of in each case at least one dicarboxylic acid with in each case at least one polyhydroxyl compound,
b) reaction of the base polyesterol from a) (or a mixture of the base polyesterols from a))
with at least one enzyme and, if appropriate, with further polyhydroxyl compounds.

The two-stage preparation of the polyesterols according to the invention comprising an actual polycondensation step a) with elimination of water and an enzymatically catalyzed transesterification and/or glycolysis step b) has the clear advantage that frequent starting material and product changes in the esterification reactor or incomplete utilization of capacity can be avoided in the preparation of relatively small quantities. The transesterification and/or glycolysis in process step b) is carried out in reactors which require less infrastructure. The temperature range 50-120° C. in particular is more readily attainable in industry. In addition, the transesterification does not require removal of water by means of reduced pressure, inert gas or entrainers. This process thus offers the advantages that the utilization of the capacity of classical production plants can be improved by avoidance of product changes and insufficient utilization of the reactor volume in the preparation of relatively small quantities of special polyesterols can be avoided. These advantages lead to a greatly reduced logistic and economic outlay and thus finally also to a lower price for special polyesterols. The process of the invention has the further advantage that it produces polyesterols having low acid numbers which are distinctly more suitable than polyesterols having high acid numbers for the preparation of many polyurethanes. However, a prerequisite for this is that base polyesterols, enzymes and, if appropriate, further polyhydroxyl compounds which together have a water content of less than 0.1% by weight, preferably less than 0.05% by weight, more preferably less than 0.03% by weight, in particular less than 0.01% by weight, are used in process step b).

Although processes in which polyesters are prepared by lipase-catalyzed “transesterification reactions”, similarly to process step b), are already known from the prior art, these processes are generally “single-stage transesterifications” starting from previously polycondensed starting materials, i.e. these processes do not comprise a preceding polycondensation step such as step a) according to the invention. Furthermore, some of the transesterification processes of the prior art are transesterifications of polyesters of the AB type (instead of transesterifications of polyesters of the AA-BB type as in process step b)). In addition, the previously known transesterification processes are generally carried out exclusively in the presence of a solvent, while the transesterification step b) according to the invention can be carried out either in the presence or in the absence of a solvent. By contrast, the transesterification step b) according to the invention is even preferably carried out in the absence of any solvent (i.e. “in bulk”).

The abovementioned single-stage lipase-catalyzed transesterifications of the prior art will be discussed briefly below.

Takamoto et al., Macromol. Biosci. 1, 223 (2001) describe the transesterification of poly-ε-caprolactone and polybutanediol adipate in the solvent toluene using a lipase from Candida antartica. ¹³C-NMR analyses of the process products show that the effectiveness of the transesterification is dependent on the type of acid or diol components used, on the choice and amount of solvent and also on the reaction time. In the case of the reaction of polybutanediol adipate with poly-ε-caprolactone in toluene, random copolymers were able to be achieved after a reaction time of about 168 hours.

WO 98/55642 describes a lipase-catalyzed process for preparing polyesterols. Mention is made, inter alia, of the possibility of not only monomers but also prefabricated polyester alcohols or polyesterdicarboxylic acids being able to be incorporated in the form of entire polymer blocks into a “growing polyester” without said polymer blocks being transesterified to form random polymers as would be the case in the classical solvent- and catalyst-dependent high-temperature processes for preparing polyesters (see page 8, last line, to page 9, line 10, of WO 98/55642). It can thus be concluded from this statement that no transesterification reactions can in general take place under the conditions of the enzymatic synthesis as disclosed in WO 98/55642.

McCabe et al., Tetrahedron 60 (2004), 765-770, describe the influence of the solvent used on the mechanism of the enzymatic transesterification of polyesters. It is stated, inter alia, that polyesters which have been prepared in the absence of solvents have different properties than polyesters which have been prepared in the presence of solvents. For example, polyesters having higher molecular weights and having a lower polydispersity can be prepared in the absence of solvents. Here, the expression “polymers having a low polydispersity” refers to a polymer mixture having uniform degrees of polymerization or a polymer mixture whose individual polymer chains have a low band width of different degrees of polymerization.

Consequently, polyesters which have been prepared by solvent-free enzymatic processes should have the advantage that they generally have higher molecular weights, are more uniform in terms of their molecular weight distribution and would therefore in some cases be expected to be superior in terms of their physical properties over conventionally prepared polyesters. However, the above-discussed prior art generally expresses the opinion that virtually no transesterification reactions take place in solvent-free enzymatic processes. A reason for this assumption could be that, according to general technical knowledge, most enzymes can display their full reactivity only in the presence of a solvent, in particular in the presence of water. Thus, none of the above-cited documents of the prior art discloses the possibility of transesterification of polyesterols in the absence of a solvent (or in bulk).

Only in Kumar et al., J. Am. Chem. Soc. 122 (2000), 11767, is a solvent-free process for the transesterification of two polyesters of the AB type, namely poly-ε-caprolactone having a molecular weight of 9200 g/mol and poly(ω-pentadecalactone) having a molecular weight of 4300 g/mol, by means of Novozym 435 at 70-75° C. described (transesterification in bulk). The microstructure of the transesterification product of Kumar et al., which was examined by means of ¹³C-NMR, showed that a random copolymer was obtained after just one hour. Nevertheless, Kumar et al. disclose only the possibility of a transesterification of polyesters of the AB type, but a two-stage process for preparing special polyesterols of the AA-BB type, which leads to a large number of special polyesterols having low acid numbers and having slightly different specific properties without costly starting material and product changes is not disclosed in Kumar et al.

Furthermore, the transesterification of Kumar et al. takes place at a relatively high total water content (namely in the range from 0.8% by weight to 1.5% by weight). Such high water contents generally result in formation of polyesterols which have high acid numbers of above 10 mg KOH/g and are distinctly less suitable for the preparation of polyurethane than are polyesterols having low acid numbers of less than 3 mg KOH/g, preferably less than 2 mg KOH/g, in particular less than 1 mg KOH/g. This is confirmed, in particular, by comparative example D1 in which ethylene glycol adipate having an initial acid number of 0.6 mg KOH/g and diethylene glycol adipate having an initial acid number of 0.8 mg KOH/g are reacted at a relatively high water content of 0.5% by weight (in Kumar et al., the water content is greater than 0.8% by weight). The transesterification product formed had an acid number of 45 mg KOH g and would thus be expected to be unsuitable or only poorly suitable for polyurethane production because of its high acid number (see also comparative example D2). Polyesterols having high acid numbers tend, as mentioned above, to give a relatively low molecular weight and to result in undesirable bubble formation due to the formation of gaseous carbon dioxide during the polyurethane reaction.

In the first step (step a)) of the two-stage process of the invention for preparing polyesterols, only a few base polyesterols are prepared by standard methods, preferably by means of high-temperature polycondensation, more preferably by means of high-temperature polycondensation aided by an esterification catalyst. The base polyols formed are then converted in the second step into virtually any desired number of different special polyesterols by enzymatic transesterification and/or glycolysis without a costly starting material/product change being necessary. In particular, the complicated and costly step of high-temperature condensation (high temperatures, need for an esterification catalyst, etc.) is restricted to the production of only a few base polyesterols as a result of this two-stage production process.

However, the first process step can, as an alternative, also be carried out by means of an enzymatic polycondensation instead of a high-temperature polycondensation aided by an esterification catalyst. In the enzymatic polycondensation, preference is given to using a lipase or hydrolase, preferably a lipase, in particular one of the lipases Candida antartica, Candida cylinderacea, Mucor meihei, Pseudomonas cepacia, Pseudomonas fluorescens, Burkholderia plantarii, at 20-110° C., preferably at 50-90° C. The enzymes can also be immobilized on a support material.

If a high-temperature polycondensation is carried out in process step a), which is preferred to the enzymatic polycondensation in step a), an organic metal compound, e.g. titanium tetrabutoxide, tin dioctoate or dibutyltin dilaurate, or an acid such as sulfuric acid, p-toluenesulfonic acid or a base such as potassium hydroxide or sodium methoxide is preferably used as esterification catalyst. This esterification catalyst is generally homogeneous and generally remains in the polyesterol after the reaction is complete. The high-temperature polycondensation is carried out at 160-280° C., preferably at 200-250° C.

In the preparation of the at least one base polyesterol according to step a) by means of a conventional high-temperature polycondensation or by means of an enzymatic polycondensation, the water liberated in the condensation reaction is preferably removed continuously.

As dicarboxylic acid, preference is given to using adipic acid or other aliphatic dicarboxylic acids, terephthalic acid or other aromatic dicarboxylic acids. Suitable polyhydroxyl compounds are all at least dihydric alcohols, but preferably diol components such as ethylene glycol, diethylene glycol, 1,3-propanediol, 1,2-propanediol, 1,4-butanediol, 1,5-pentanediol, 1,6-hexanediol, 2-methyl-1,3-propanediol, 3-methyl-1,5-pentanediol.

Process step a) can be carried out either in the presence of a solvent or else in the absence of a solvent, i.e. in bulk, regardless of whether a high-temperature polycondensation (aided by means of an esterification catalyst) or an enzymatically catalyzed polycondensation is carried out. However, preference is given to carrying out process step a) in bulk, i.e. in the absence of any solvent.

The base polyesterols prepared in step a) are chosen according to the desired properties of the end products. Base polyesterols which are preferably used are polyesterols based on adipic acid and a diol component, preferably ethylene glycol, diethylene glycol, 1,3-propanediol, 1,2-propanediol, 1,4-butanediol, 1,5-pentanediol, 1,6-hexanediol, 2-methyl-1,3-propanediol, 3-methyl-1,5-pentanediol.

The preferred molecular weight of the base polyesterols prepared in step a) is in the range from 200 g/mol to 10 000 g/mol, particularly preferably in the range 500-5000 g/mol.

The acid numbers of the base polyesterols prepared in step a) are preferably in the range below 3 g KOH/kg, more preferably in the range below 2 g KOH/kg, in particular in the range below 1 g KOH kg. The acid number serves to indicate the content of free organic acids in the polyesterol. The acid number is determined by the number of mg of KOH (or g of KOH) consumed in the neutralization of 1 g (or 1 kg) of the sample.

The functionality of the base polyesterols prepared in step a) is preferably in the range from at least 1.9 to 4.0, more preferably in the range from 2.0 to 3.0. The hydroxyl number (hereinafter referred to as OHN for short) of the base polyesterols prepared in step a) is calculated from the number average molecular weight Mₙ and the functionality f of the polyesterol according to the formula

OHN=56100*f/Mₙ.

According to the invention, it has surprisingly been able to be shown that the enzymatic transesterification according to step b) is also possible for base polyesterols which originate from classical high-temperature catalysis in step a) and thus already have a relatively high mean molecular weight (for example 3000 g/mol) and consequently also low acid numbers. It has long been known that polyesterols which have high mean molecular weights and consequently low acid numbers, in particular, have little tendency if any to undergo transesterification (cf. 2nd section by McCabe and Taylor, Tetrahedron 60 (2004), 765-770).

The second process step (step b)) is carried out exclusively enzymatically. Step b) comprises either
1. enzyme-catalyzed transesterification (without additional glycolysis),
2. enzyme-catalyzed glycolysis (without additional transesterification) or
3. a mixed reaction comprising enzyme-catalyzed transesterification and enzyme-catalyzed glycolysis or alcoholysis.

In the enzyme-catalyzed transesterification (cf. No. 1), two or more base polyesterols from step a) are reacted with a sufficient amount of suitable enzymes without any additional polyhydric polyhydroxy compounds (diols, glycols) being added. In this case, a new polyesterol which in the ideal case is a random copolymer of the monomers of all base polyesterols used is formed.

In the enzyme-catalyzed glycolysis, only one base polyesterol from step a) is reacted with one or more polyhydroxy compounds, preferably with diols or polyols, and a suitable amount of the enzyme. In this case, the mean molecular weight of the base polyesterol is generally reduced by glycolysis or alcoholysis of part of the ester bonds.

As an alternative, a mixed reaction comprising enzyme-catalyzed transesterification and enzyme-catalyzed glycolysis or alcoholysis can take place in process step b). Here, a mixture of at least two base polyesterols from step a) and at least one polyhydric polyhydroxy compound (preferably diols or polyols) is reacted with a suitable amount of the enzyme. In this variant of process step b), the change in the mean molecular weight or the other properties (viscosity, acid number, melting point, etc.) of the base polyesterols depends on the components used in the individual case, in particular on the type and amount of the base polyesterol(s) used and on the type and amount of the polyhydroxy compounds used.

The properties of the end product (the polyesterol) likewise depend on whether the transesterification or glycolysis according to step b) has proceeded to completion. The completeness of the transesterification or glycolysis according to step b) in turn depends on the reaction time, with long reaction times ensuring complete transesterification or glycolysis. The reaction times for the transesterification step b) are preferably selected so that polyesterols which have very similar properties to polyesterols which have been prepared by the classical single-stage high-temperature polycondensation process are obtained in the end. The reaction time for the transesterification or glycolysis according to step b) can thus be from 1 to 36 hours, preferably from 2 to 24 hours, in each case depending on the amount and identity of the enzyme used for the reaction.

The enzymatic transesterification or glycolysis is carried out using a lipase or hydrolase, preferably a lipase, particularly preferably one of the lipases Candida antartica, Candida cylinderacea, Mucor meihei, Pseudomonas cepacia, Pseudomonas fluorescens, Burkholderia plantarii, at 20-110° C., preferably 30-90° C., more preferably 50-80° C., in particular 70° C. The lipases Candida antartica and Burkholderia plantarii are particularly suitable for the enzymatic transesterification or glycolysis of the base polyesterols in step b). The enzyme Candida antartica is commercially available in immobilized form on a macroporous acrylic resin as “Novozym 435®” or in soluble form as “Novozym 525”. The use of “Novozym 435®” and “Novozym 525” in process step b) is thus particularly preferred.

The enzymes used can thus also be immobilized on a support material. As support materials, it is possible to use all suitable materials, but preferably solid materials having large surface areas, more preferably resins, polymers, etc., on which the enzymes can be present in preferably covalently bound form. Particular preference is given to using resin beads having a small diameter as support material. After the esterification and/or glycolysis reaction of process step b) is complete, the enzymes are, if they have been immobilized on a support material, preferably separated off from the polyesterol. This separation can be achieved, for example, by means of classical separation methods such as filtration, centrifugation or the like which exploit the different particle sizes or the different particle weights. In the case of magnetic support materials, for example, the separation can also be carried out via the use of magnetic forces. The removal of the enzymes immobilized on support materials after the end of the process step b) prevents these from interfering in the use of the polyesterols prepared, in particular in further reactions of these polyesterols, e.g. in the reaction of the polyesterols with isocyanates to form polyurethanes.

If soluble enzymes which have not been immobilized on support materials are used in process step b), it is generally not necessary to separate these from the polyesterol. In this case, it is frequently sufficient to inactivate the enzymes after the transesterification or glycolysis in process step b). The inactivation of the enzymes can be achieved by means of a wide variety of methods which lead to denaturation of the enzyme, e.g. the inactivation of soluble enzymes by means of chemical substances, but preferably inactivation of the enzymes by means of simple thermal denaturation at high temperatures. Preference is given to employing temperatures above 110° C., more preferably above 150° C., for the thermal denaturation.

The reaction of process step b) can, like process step a), be carried out in the presence of a solvent or in the absence of a solvent (reaction “in bulk”).

If the reaction of process step b) is carried out in the presence of a solvent, it is possible to use all known suitable solvents, in particular the solvents toluene, dioxane, hexane, tetrahydrofuran, cyclohexane, xylene, dimethyl sulfoxide, dimethylformamide, N-methylpyrrolidone, chloroform. The choice of solvent depends on the starting materials (the base polyesterols and the polyhydroxy compounds) used in the particular case and, in particular, on their solublity properties. However, the reaction of process step b) in the presence of a solvent has the disadvantage that it comprises additional process substeps, namely the dissolution of the at least one base polyesterol in the solvent and the removal of the solvent after the reaction. Furthermore, the dissolution of the at least one base polyesterol in the solvent can, depending on the hydrophobicity properties of the base polyesterol, be problematical and may decrease the yield.

However, in a preferred embodiment of the process, the reaction of step b) is carried out in the absence of a solvent (also referred to as “reaction in bulk”). If base polyesterols having a high molecular weight are to be subjected to the enzymatic esterification according to step b), the effectiveness of this transesterification reaction is limited by the low solubility of these base polyesterols of high molecular weight in most solvents. On the other hand, the number of hydroxyl groups of the solvent has only a small influence on the effectiveness of the transesterification reaction. Thus, for example, according to McCabe and Taylor, Tetrahedron 60 (2004), 765-770, no esterification reaction takes place in 1,4-butanediol as solvent even though the concentration of hydroxyl groups is very high. In contrast, transesterification does take place in polar solvents (dioxane, toluene).

In a further preferred embodiment of the process, process step b) is preferably carried out using base polyesterols, enzymes and, if appropriate, additional polyhydroxyl compounds which together have a water content of less than 0.1% by weight, preferably less than 0.05% by weight, more preferably less than 0.03% by weight, in particular less than 0.01% by weight. In the case of higher water contents during process step b), hydrolysis also takes place alongside the transesterification, so that the acid number of the polyesterol would increase in an undesirable way during step b). Carrying out step b) of the process of the invention at a water content of less than 0.1% by weight, preferably less than 0.05% by weight, more preferably less than 0.03% by weight, in particular less than 0.01% by weight, thus leads to the preparation of special polyesterols having a low acid number as end products. Polyesterols having a low acid number are generally more stable toward hydrolysis than polyesterols having a high acid number, since free acid groups catalyze the reverse reaction, i.e. hydrolysis.

Preparation of polyesterols having water contents above 0.1% by weight leads to polyesterols having an acid number of greater than 10 mg KOH/g (cf. comparative examples D1 and D2). However, polyesterols having such high acid numbers (greater than 10 mg KOH/g) are unsuitable or have only poor suitability for most industrial applications, in particular for use in the preparation of polyesterols.

Depending on the atmospheric humidity, enzymes can have water contents of >0.1% by weight. For this reason, drying of the enzyme is necessary before use of the enzyme in the transesterification reaction of process step b). Drying of the enzyme is carried out by the customary drying methods, e.g. drying in a vacuum drying oven at temperatures of 60-120° C. under a pressure of from 0.5 to 100 mbar or by suspending the enzyme in toluene and subsequently distilling off the toluene under reduced pressure at temperatures of 50-100° C.

Polyesterols, too, take up at least 0.01%, but generally at least 0.02%, in some cases even more than 0.05%, of water, depending on the atmospheric humidity and temperature. Depending on the degree of conversion and molecular weight of the base polyesterols used, this water concentration is higher than the equilibrium water concentration. If the polyesterol is not dried before process step b), hydrolysis of the polyesterol inevitably occurs.

The water content of the base polyesterols used in step b) are therefore preferably dried prior to the transesterification in process step b). The enzyme to be used in step b) and any polyhydric polyhydroxyl compound to be used (e.g. the diol) are also preferably dried prior to the transesterification reaction in order to achieve the abovementioned low water content in the transesterification. Drying can be carried out using customary drying methods of the prior art, for example by drying over molecular sieves or by means of a falling film evaporator. As an alternative, base polyesterols having low water contents (preferably less than 0.1% by weight, more preferably less than 0.05% by weight, even more preferably less than 0.03% by weight, in particular less than 0.01% by weight) can also be obtained by carrying out the reaction according to process step a) and also any intermediate storage of the at least one base polyesterol entirely under inert conditions, for example in an inert gas atmosphere, preferably in a nitrogen atmosphere. In this case, the base polyesterols have no opportunity of taking up relatively large amounts of water from the environment right from the beginning. A separate drying step could then become superfluous.

In a further preferred embodiment of the process, the at least one base polyesterol from process step a) is therefore temporarily stored, preferably in an inert gas atmosphere, so as to keep the water content low prior to the reaction according to process step b). A mixture of two or more base polyesterols in an appropriate ratio can then be made up from the temporarily stored base polyesterols in order to obtain a particular special polyesterol having very specific physical properties and a specific structure after the transesterification (and after any additional glycolysis by means of polyhydroxy compounds).

The invention further provides a polyesterol which has been prepared or is obtainable by one of the above-described two-stage processes comprising the process steps a) and b). These polyesterols according to the invention generally have relatively low acid numbers, preferably acid numbers of less than 3 mg KOH per gram of polyesterol, more preferably less than 2 mg KOH per gram of polyesterol, in particular less than 1 mg KOH per gram of polyesterol.

These low acid numbers are, in particular, achieved by process step b) being carried out at a water content of preferably less than 0.1% by weight, more preferably less than 0.05% by weight, even more preferably less than 0.03% by weight, in particular less than 0.01% by weight.

Process step a) can be carried out using all reactors whose use is known for classical high-temperature polycondensations or for enzymatic polycondensations (cf. Ullmann Encyclopedia (Electronic Release), chapter: Polyesters, paragraph: Polyesters as Intermediaries for Polyurethane). A stirred tank reactor with stirrer and distillation column is preferably used for carrying out process step a). This apparatus is generally a closed system and can generally be evacuated by means of a vacuum pump. The starting materials are heated with stirring and preferably with exclusion of air (e.g. in a nitrogen atmosphere or under reduced pressure). The water formed in the polycondensation is preferably distilled off at a low pressure or a continually decreasing pressure (cf. batchwise vacuum melt process, Houben-Weyl 14/2, 2).

In the purge gas melt process (cf. BASF, NL 6 505 683, 1965), the products which can be distilled off, e.g. the water of reaction, are not removed by decreasing the pressure but instead by passing an inert gas such as nitrogen or carbon dioxide through the reaction mixture.

In the azeotropic process (H. Batzer, Makromol. Chem. 7 (1951) 8), the polycondensation is carried out at atmospheric pressure in the presence of an inert solvent as entrainer (e.g. in the presence of toluene or xylene), with the aid of which the water of reaction being formed is removed. For this purpose, the apparatus has to have additional facilities which allow the removal and continuous recycling of the entrainer.

As an alternative, continuous esterification reactors, as are used, for example, for the preparation of thermoplastic polyesters such as PET and PBT, can also be used for this process step a) (cf. Ullmann, chapter: Polyesters, paragraph: Thermoplastic Polyesters (Production)).

The reactor material has to be corrosion-resistant, heat-resistant and also acid-resistant. These requirements are met, for example, by austenitic chromium-nickel-molybdenum alloys (e.g. V4A steel DIN1.4571).

Process step b) is carried out in a temperature range of 50-120° C., preferably 60-100° C., particularly preferably 70-90° C., under atmospheric pressure. The reaction is preferably carried out in an inert atmosphere with exclusion of atmospheric moisture, for example by passing nitrogen over the reaction mixture. Process step b) is carried out in a heated stirred tank reactor. However, the process of the invention can also be carried out batchwise, semicontinuously or continuously in conventional bioreactors. Suitable modes of operation and reactors are known to those skilled in the art and are described, for example, in Römpp Chemie Lexikon, 9th edition, Thieme Verlag, keyword “Bioreaktor” and “Festbettreaktor” or Ullmanns's Encyclopedia of Industrial Chemistry, Electronic Release, under the keyword “Bioreactors” (similar to WO 03/042227, p. 5, line 33).

The present invention is illustrated by the following examples.

### A. EXAMPLES OF THE GLYCOLYSIS OF POLYESTEROLS

In all the following examples of the glycolysis of polyesterols, identical polyesterols derived from adipic acid and 1,4-butanediol (1,4-butanediol adipate) having a mean molecular weight of 5000 g/mol, a base number (hereinafter referred to as “OHN”) of 23.5 mg KOH/g and an acid number (hereinafter referred to as “AN”) of 1.6 mg KOH g were used in each case.

These 1,4-butanediol adipates were each prepared as follows (process step a)) for all examples and comparative examples of the glycolysis of polyesterols:

Preparation of Polybutanediol Adipate by Means of High-Temperature Poly-Condensation:

47.4 kg of 1,4-butanediol were placed in a 250 l stirred tank reactor provided with a column and a stirrer. At 90° C., 68.9 kg of adipic acid were added via a pot. The reaction mixture was heated at 40° C./h to 240° C. The water of reaction formed was removed from the reactor by distillation. After a reaction time of 3 hours, the reactor pressure was reduced from atmospheric pressure to 30-50 mbar. After a reaction time of 48 hours, the acid number of the polyesterol prepared according to step a) was 1.6 mg KOH/g, the OH number was 23.5 mg KOH/g, and the water content immediately after the end of the reaction was <0.01% by weight.

### Comparative Example A1

### Transesterification of Polyesterols with Diols at 90° C. Without Catalyst

450 g of a 1,4-butanediol adipate (OHN=23.5 mg KOH/g, AN=1.6 mg KOH/g) were placed in a three-necked flask provided with a stirrer, reflux condenser and nitrogen inlet. The polyesterol was dried under reduced pressure (15 mbar) at the reaction temperature for about 30 minutes.

The polyesterol was heated to a reaction temperature of 90° C. After the reaction temperature had been reached, 34 g of butanediol were added via a dropping funnel which had been heated to the reaction temperature. To determine the progress of the reaction, the acid number, OH number, the water content and the viscosity were measured as a function of the reaction time (cf. table 1).

TABLE 1

| | Reaction | | | Viscosity | OHN |
|---|---|---|---|---|---|
| Temperature | time | AN | Water | at 75° C. | [mg |
| [° C.] | [minutes] | [mg KOH/g] | content | [mPas] | KOH/g] |
| 90 | 15 | 1.5 | 0.03 | 2850 | — |
| 90 | 30 | 1.4 | 0.03 | 3140 | 104 |
| 91 | 120 | 1.5 | 0.03 | 3170 | 107 |
| 89 | 180 | 1.4 | 0.03 | 3200 | — |
| 90 | 240 | 1.4 | 0.03 | 3180 | 106 |
| 91 | 300 | 1.5 | 0.03 | 3220 | — |
| 90 | 360 | 1.5 | 0.03 | 3280 | 105 |
| 90 | 420 | 1.5 | 0.03 | 3100 | 105 |

The viscosity, which is a measure of the weight average molecular weight, remained constant during the reaction. Thus, the distribution had not been made more uniform and thus no reaction between the components had taken place.

### Comparative Example A2

### Transesterification of Polyesterols with Diols at 200° C. Without Catalyst

450 g of a 1,4-butanediol adipate (OHN=23.5 mg KOH/g, AN=1.6 mg KOH/g) were placed in a three-necked flask provided with a stirrer, reflux condenser and nitrogen inlet. The polyesterol was dried at 90° C. under reduced pressure (15 mbar) for about 30 minutes.

The polyesterol was heated to a reaction temperature of 200° C. After the reaction temperature had been reached, 34 g of butanediol were added via a dropping funnel which had been heated to the reaction temperature. To determine the progress of the reaction, the acid number, OH number, the water content and the viscosity were measured as a function of the reaction time (cf. table 2).

TABLE 2

| | Reaction | | | Viscosity | OHN |
|---|---|---|---|---|---|
| Temperature | time | AN | Water | at 75° C. | [mg |
| [° C.] | [minutes] | [mg KOH/g] | content | [mPas] | KOH/g] |
| 197 | 5 | 1.4 | 0.03 | 2060 | 150 |
| 202 | 15 | 1.4 | 0.05 | 1180 | 150 |
| 204 | 30 | 1.4 | 0.04 | 736 | 149 |
| 203 | 60 | 1.4 | 0.05 | 406 | 150 |
| 201 | 120 | 1.4 | 0.05 | 236 | 149 |
| 201 | 180 | 1.5 | 0.04 | 149 | 149 |
| 202 | 240 | 1.5 | 0.05 | 147 | 148 |
| 201 | 300 | 1.6 | 0.05 | 147 | 148 |

The viscosity, which is a measure of the weight average molecular weight, decreases continuously. The molecular weight distribution becomes more uniform and a reaction between the components thus takes place. The end point of the reaction can be recognized from the reaching of a plateau after about 180 minutes.

### Example A3

### Enzymatic Glycolysis of Polyesterols with Diols Using 1% of Novozym 435 at 90° C.

450 g of a 1,4-butanediol adipate (OHN=23.5 mg KOH g, AN=1.6 mg KOH/g) were placed in a three-necked flask provided with a stirrer, reflux condenser and nitrogen inlet. The polyesterol was dried under reduced pressure (15 mbar) at the reaction temperature for about 30 minutes. After drying was complete, 5.2 g of dried Novozym were added (corresponds to 1% by weight).

To dry the Novozym 435, a 30% suspension of Novozym 435 in toluene was prepared in a 100 ml flask. Immediately before commencement of the reaction, the toluene was removed by distillation at about 50-60° C. under reduced pressure (100 mbar) on a rotary evaporator.

The mixture comprising Novozym 435 and polyesterol was heated to a reaction temperature of 90° C. After the reaction temperature had been reached, 52 g of butanediol were added via a dropping funnel which had been heated to the reaction temperature. To determine the progress of the reaction, the acid number (AN), the OH number (OHN), the water content and the viscosity were measured as a function of the reaction time (table 3).

TABLE 3

| | Reaction | | | Viscosity | OHN |
|---|---|---|---|---|---|
| Temperature | time | AN | Water | at 75° C. | [mg |
| [° C.] | [minutes] | [mg KOH/g] | content | [mPas] | KOH/g] |
| 89 | 5 | 1.3 | 0.05 | 2980 | — |
| 90 | 15 | 1.3 | 0.05 | 1930 | — |
| 91 | 30 | 1.2 | 0.05 | 1580 | 152 |
| 89 | 60 | 1.1 | 0.05 | 1160 | 152 |
| 90 | 135 | 1.0 | 0.05 | 645 | 153 |
| 90 | 255 | 1.0 | 0.05 | 364 | 152 |
| 90 | 315 | 1.0 | 0.05 | 300 | 152 |
| 90 | 1500 | 0.9 | 0.05 | 160 | 152 |

After 25 hours (1500 minutes), a viscosity of 160 mPas was reached; this corresponded to the viscosity of the plateau value of the product transesterified at 200° C. The products from example A2 and example A3 could thus be regarded as identical.

### Example A4

### Enzymatic Glycolysis of Polyesterols with Diols using 5% Novozym 435 at 90° C.

450 g of a 1,4-butanediol adipate (OHN=23.5 mg KOH/g, AN=1.6 mg KOH g) were placed in a three-necked flask provided with a stirrer, reflux condenser and nitrogen inlet. The polyesterol was dried under reduced pressure (15 mbar) at the reaction temperature for about 30 minutes. After drying was complete, 25.1 g of dried Novozym were added (corresponds to 5% by weight).

To dry the Novozym 435, a 30% suspension of Novozym 435 in toluene was prepared in a 100 ml flask. Immediately before commencement of the reaction, the toluene was removed by distillation at about 50-60° C. under reduced pressure (100 mbar) on a rotary evaporator.

The mixture comprising Novozym 435 and polyesterol was heated to a reaction temperature of 90° C. After the reaction temperature had been reached, 52 g of butanediol were added via a dropping funnel which had been heated to the reaction temperature. To determine the progress of the reaction, the acid number (AN), the OH number (OHN), the water content and the viscosity were measured as a function of the reaction time (cf. table 4).

TABLE 4

| | Reaction | | | Viscosity | OHN |
|---|---|---|---|---|---|
| Temperature | time | AN | Water | at 75° C. | [mg |
| [° C.] | [minutes] | [mg KOH/g] | content | [mPas] | KOH/g] |
| 89 | 10 | 1.6 | 0.05 | 1810 | — |
| 90 | 20 | 1.2 | 0.05 | 1040 | — |
| 91 | 30 | 1.2 | 0.05 | 710 | 147 |
| 89 | 60 | 1.2 | 0.05 | 360 | 146 |
| 90 | 120 | 1.5 | 0.05 | 194 | 148 |
| 90 | 180 | 1.3 | 0.05 | 159 | 148 |
| 90 | 240 | 1.2 | 0.05 | 142 | 147 |
| 90 | 300 | 1.4 | 0.05 | 150 | 147 |

After about 240 minutes, a viscosity of 140-150 mPas was reached; this corresponded to the viscosity of the plateau value of the product transesterified at 200° C. The products from example A2 and example A4 could thus be regarded as identical.

### Example A5

### Enzymatic Glycolysis of Polyesterols with Diols using 10% of Novozym 435 at 90° C.

450 g of a 1,4-butanediol adipate (OHN=23.5 mg KOH/g, AN=1.6 mg KOH/g) were placed in a three-necked flask provided with a stirrer, reflux condenser and nitrogen inlet. The polyesterol was dried under reduced pressure (15 mbar) at the reaction temperature for about 30 minutes. After drying was complete, 50.2 g of dried Novozym were added (corresponds to 10% by weight).

To dry the Novozym 435, a 30% suspension of Novozym 435 in toluene was prepared in a 100 ml flask. Immediately before commencement of the reaction, the toluene was removed by distillation at about 50-60° C. under reduced pressure (100 mbar) on a rotary evaporator.

The mixture comprising Novozym 435 and polyesterol was heated to a reaction temperature of 90° C. After the reaction temperature had been reached, 52 g of butanediol were added via a dropping funnel which had been heated to the reaction temperature. To determine the progress of the reaction, the acid number (AN), the OH number (OHN), the water content and the viscosity were measured as a function of the reaction time (cf. table 5).

TABLE 5

| | Reaction | | | Viscosity | OHN |
|---|---|---|---|---|---|
| Temperature | time | AN | Water | at 75° C. | [mg |
| [° C.] | [minutes] | [mg KOH/g] | content | [mPas] | KOH/g] |
| 89 | 10 | 1.5 | 0.07 | 1160 | — |
| 90 | 20 | 1.6 | 0.07 | 468 | — |
| 91 | 30 | 1.7 | 0.06 | 292 | 151 |
| 89 | 60 | 1.7 | 0.06 | 170 | 151 |
| 90 | 120 | 1.5 | 0.06 | 140 | 151 |
| 90 | 180 | 1.4 | 0.05 | 135 | 150 |
| 90 | 240 | 1.4 | 0.05 | 133 | 150 |
| 90 | 300 | 1.3 | 0.05 | 139 | 149 |

After about 120 minutes, a viscosity of 130-140 mPas was reached; this corresponded to the viscosity of the plateau value of the product transesterified at 200° C. The products from example A2 and example A5 could thus be regarded as identical.

### Example A6

### Enzymatic Glycolysis of Polyesterols with Eiols using 10% of Novozym 435 at 60° C.

450 g of a 1,4-butanediol adipate (OHN=23.5 mg KOH/g, AN=1.6 mg KOH g) were placed in a three-necked flask provided with a stirrer, reflux condenser and nitrogen inlet. The polyesterol was dried under reduced pressure (15 mbar) at the reaction temperature for about 30 minutes. After drying was complete, 52.2 g of dried Novozym were added (corresponds to 10% by weight).

To dry the Novozym 435, a 30% suspension of Novozym 435 in toluene was prepared in a 100 ml flask. Immediately before commencement of the reaction, the toluene was removed by distillation at about 50-60° C. under reduced pressure (100 mbar) on a rotary evaporator.

The mixture comprising Novozym 435 and polyesterol was heated to a reaction temperature of 60° C. After the reaction temperature had been reached, 52 g of butanediol were added via a dropping funnel which had been heated to the reaction temperature. To determine the progress of the reaction, the acid number (AN), the OH number (OHN), the water content and the viscosity were measured as a function of the reaction time (cf. table 6).

TABLE 6

| | Reaction | | | Viscosity | OHN |
|---|---|---|---|---|---|
| Temperature | time | AN | Water | at 75° C. | [mg |
| [° C.] | [minutes] | [mg KOH/g] | content | [mPas] | KOH/g] |
| 61 | 15 | 1.4 | 0.08 | 2010 | — |
| 60 | 30 | 1.5 | 0.07 | 1500 | — |
| 61 | 60 | 1.7 | 0.08 | 611 | 139 |
| 61 | 90 | 1.8 | 0.08 | 358 | 141 |
| 61 | 120 | 1.7 | 0.08 | 260 | 144 |
| 62 | 240 | 1.9 | 0.08 | 160 | 146 |
| 60 | 300 | 1.9 | 0.08 | 140 | 147 |

After about 240 minutes, a viscosity of 140-160 mPas was reached; this corresponded to the viscosity of the plateau value of the product transesterified at 200° C. The products from example A2 and example A6 could thus be regarded as identical.

### B. EXAMPLES OF THE TRANSESTERIFICATION OF POLYESTEROLS

In all the following examples of the transesterification of polyesterols, identical polyesterols derived from adipic acid and ethylene glycol (polyethylene glycol adipate) and from adipic acid and 1,4-butanediol (polybutanediol adipate) were used in each case. The polyethylene glycol adipate had a mean molecular weight of 1000 g/mol, a base number (hereinafter referred to as “OHN”) of 99.3 mg KOH g and an acid number (hereinafter referred to as “AN”) of 2.4 mg KOH/g. The polybutanediol adipate had a mean molecular weight of 5000 g/mol, a base number of 23.5 mg KOH/g and an acid number of 1.6 mg KOH/g.

The polyethylene glycol adipates and the polybutanediol adipates were each prepared as follows for all the following examples and comparative examples of the transesterification of polyesterols (process step a)):

Preparation of Polybutanediol Adipate:

47.4 kg of 1,4-butanediol were placed in a 250 l stirred tank reactor provided with a column and a stirrer. At 90° C., 68.9 kg of adipic acid were added via a pot. The reaction mixture was heated at 40° C./h to 240° C. The water of reaction formed was removed from the reactor by distillation. After a reaction time of 3 hours, the reactor pressure was reduced from atmospheric pressure to 30-50 mbar. After a reaction time of 48 hours, the acid number of the polyesterol prepared according to step a) was 1.6 mg KOH/g, the OH number was 23.5 mg KOH/g, and the water content immediately after the end of the reaction was <0.01% by weight.

Preparation of Polyethylene Glycol Adipate:

39.6 kg of ethylene glycol were placed in a 250 l stirred tank reactor provided with a column and a stirrer. At 90° C., 80.2 kg of adipic acid were added via a pot. The reaction mixture was heated at 40° C./h to 240° C. The water of reaction formed was removed from the reactor by distillation. After a reaction time of 3 hours, the reactor pressure was reduced from atmospheric pressure to 30-50 mbar. After a reaction time of 24 hours, the acid number of the polyesterol prepared according to step a) was 2.4 mg KOH/g, the OH number was 99.8 mg KOH/g, and the water content immediately after the end of the reaction was <0.01% by weight.

### Example B1

### Enzymatic Transesterification of Polyesterols Using 1% of Novozym 435 at 90° C.

250 g of an ethylene glycol adipate (OHN=99.3 mg KOH/g, AN=1.6 mg KOH/g) and 250 g of a 1,4-butanediol adipate (OHN=23.5 mg KOH/g, AN=2.4 mg KOH/g) were mixed by stirring in a three-necked flask provided with a stirrer, reflux condenser and nitrogen inlet. The mixture was heated to 90° C. and evacuated for 15 minutes. After admission of nitrogen, 5 g of dried Novozym 435 were added to the reaction mixture.

The drying of the Novozym 435 was carried out by preparing a 30% suspension of Novozym 435 in toluene and subsequently removing the solvent at 50-60° C. and a pressure of about 100 mbar on a rotary evaporator.

The reaction was carried out at 90° C. for 24 hours. To characterize the samples, samples were characterized by means of gel permeation chromatography at regular intervals. The polydispersity index PD=M_{w}/Mₙ (M_{w}=weight average molecular weight, Mₙ=number average molecular weight) was employed as a measure of the progress of the transesterification (cf. table 7).

TABLE 7

| | | | | Polydispersity |
|---|---|---|---|---|
| Temperature | Reaction time | AN | Water | index |
| [° C.] | [minutes] | [mg KOH/g] | content | [M_{w}/Mₙ] |
| 89 | 15 | 2.1 | 0.03 | 3.6 |
| 92 | 30 | 1.9 | 0.02 | 3.7 |
| 93 | 60 | 1.9 | 0.01 | 3.7 |
| 93 | 90 | 1.8 | 0.01 | 3.6 |
| 89 | 120 | 1.6 | 0.01 | 3.5 |
| 89 | 240 | 1.3 | 0.01 | 3.1 |
| 92 | 360 | 1.1 | 0.01 | 2.9 |
| 91 | 1440 | 0.8 | 0.01 | 2.1 |

After about 24 hours, the polydispersity index (PD) had reached a value of 2.1. This value corresponded approximately to the theoretical predictions of Flory-Schulz for an equilibrium distribution (PD=2.0) and consequently indicated that the two starting polyesterols (base polyesterols) had reacted to form a new polyesterol or that the transesterification had proceeded to completion.

The microstructure of the end product was determined by means of ¹³C-NMR. Here, the splitting of the carbon atom located in the α position relative to the carboxyl carbon of adipic acid was examined. The following ¹³C signals were observed: the signal at 24.33 ppm could be assigned to the butanediol-adipic acid-butanediol (BAB) triads. The ethylene glycol-adipic acid-ethylene glycol (EAE) triads appeared at 24.17 ppm. The signals at 24.27 ppm and 24.23 ppm corresponded to the butanediol-adipic acid-ethylene glycol (BAE) or ethylene glycol-adipic acid-butanediol (EAB) triads. In the starting polyesters, only signals which could be assigned to the corresponding homopolymers (butanediol adipate: 24.33 ppm and ethylene glycol adipate at 24.17 ppm) were detected. The end product had the ratio of the triads BAB:(EAB+BAE):EAE=28:47:25 to be expected for a random copolymer.

### Example B2

### Enzymatic Transesterification of Polyesterols Using 5% of Novozym 435 at 90° C.

250 g of an ethylene glycol adipate (OHN=99.3 mg KOH/g, AN=1.6 mg KOH/g) and 250 g of a 1,4-butanediol adipate (OHN=23.5 mg KOH/g, AN=2.4 mg KOH/g) were mixed by stirring in a three-necked flask provided with a stirrer, reflux condenser and nitrogen inlet. The mixture was heated to 90° C. and evacuated for 15 minutes. After admission of nitrogen, 25 g of dried Novozym 435 were added to the reaction mixture.

The drying of the Novozym 435 was carried out by preparing a 30% suspension of Novozym 435 in toluene and removing the solvent at 50-60° C. and a pressure of about 100 mbar on a rotary evaporator.

The reaction was carried out at 90° C. for 24 hours. To characterize the samples, samples were characterized by means of gel permeation chromatography at regular intervals. The polydispersity index PD=M_{w}/Mₙ was employed as a measure of the progress of the reaction (cf. table 8).

TABLE 8

| | | | | Polydispersity |
|---|---|---|---|---|
| Temperature | Reaction time | AN | Water | index |
| [° C.] | [minutes] | [mg KOH/g] | content | [M_{w}/Mₙ] |
| 90 | 15 | 2.2 | 0.03 | 3.6 |
| 91 | 30 | 2.2 | 0.02 | 3.4 |
| 92 | 60 | 2.1 | 0.02 | 3.0 |
| 92 | 90 | 2.0 | 0.02 | 2.7 |
| 88 | 120 | 1.7 | 0.02 | 2.6 |
| 92 | 240 | 0.6 | 0.01 | 2.3 |
| 90 | 360 | 0.1 | 0.01 | 2.1 |
| 90 | 1440 | 0.8 | 0.01 | 2.1 |

After about 360 minutes, the polydispersity index (PD) had reached a value of 2.1. This value corresponded approximately to the theoretical predictions of Flory-Schulz for an equilibrium distribution (PD=2.0) and consequently indicated that the two starting polyesterols (base polyesterols) had reacted to form a new polyesterol or that the transesterification had proceeded to completion.

The microstructure of the end product was determined by means of ¹³C-NMR. Here, the splitting of the carbon atom located in the α position relative to the carboxyl carbon of adipic acid was examined.

The following ¹³C signals were observed: the signal at 24.33 ppm could be assigned to the butanediol-adipic acid-butanediol (BAB) triads. The ethylene glycol-adipic acid-ethylene glycol (EAE) triads appeared at 24.17 ppm. The signals at 24.27 ppm and 24.23 ppm corresponded to the butanediol-adipic acid-ethylene glycol (BAE) or ethylene glycol-adipic acid-butanediol (EAB) triads. In the starting polyesters, only signals which could be assigned to the corresponding homopolymers (butanediol adipate at 24.33 ppm and ethylene glycol adipate at 24.17 ppm) were detected. The end product had the ratio of the triads BAB:(EAB+BAE):EAE=28:47:25, to be expected for a random copolymer.

### Example B3

### Enzymatic Transesterification of Polyesterols Using 10% of Novozym 435 at 90° C.

250 g of an ethylene glycol adipate (OHN=99.3 mg KOH/g, AN=1.6 mg KOH/g) and 250 g of a 1,4-butanediol adipate (OHN=23.5 mg KOH/g, AN=2.4 mg KOH/g) were mixed by stirring in a three-necked flask provided with a stirrer, reflux condenser and nitrogen inlet. The mixture was heated to 90° C. and evacuated for 15 minutes. After admission of nitrogen, 50 g of dried Novozym 435 were added to the reaction mixture.

The drying of the Novozym 435 was carried out by preparing a 30% suspension of Novozym 435 in toluene and subsequently removing the solvent at 50-60° C. and a pressure of about 100 mbar on a rotary evaporator.

The reaction was carried out at 90° C. for 24 hours. To characterize the samples, samples were characterized by means of gel permeation chromatography at regular intervals. The polydispersity index PD=M_{w}/Mₙ was employed as a measure of the progress of the reaction (cf. table 9).

TABLE 9

| | | | | Polydispersity |
|---|---|---|---|---|
| Temperature | Reaction time | AN | Water | index |
| [° C.] | [minutes] | [mg KOH/g] | content | [M_{w}/Mₙ] |
| 90 | 15 | 2.4 | 0.04 | 3.6 |
| 91 | 30 | 2.2 | 0.04 | 3.1 |
| 92 | 60 | 2.4 | 0.02 | 2.4 |
| 92 | 90 | 2.1 | 0.02 | 2.2 |
| 88 | 120 | 1.9 | 0.02 | 2.1 |
| 92 | 300 | 1.0 | 0.01 | 2.1 |
| 90 | 360 | 0.8 | 0.01 | 2.1 |
| 90 | 1440 | 0.3 | 0.01 | 2.1 |

After about 120 minutes, the polydispersity index (PD) had reached a value of 2.1. This value corresponded approximately to the theoretical predictions of Flory-Schulz for an equilibrium distribution (PD=2.0) and consequently indicated that the two starting polyesterols (base polyesterols) had reacted to form a new polyesterol or that the transesterification had proceeded to completion.

The microstructure of the end product was determined by means of ¹³C-NMR. Here, the splitting of the carbon atom located in the a position relative to the carboxyl carbon of adipic acid was examined. The following ¹³C signals were observed: the signal at 24.33 ppm could be assigned to the butanediol-adipic acid-butanediol (BAB) triads. The ethylene glycol-adipic acid-ethylene glycol (EAE) triads appeared at 24.17 ppm. The signals at 24.27 ppm and 24.23 ppm corresponded to the butanediol-adipic acid-ethylene glycol (BAE) or ethylene glycol-adipic acid-butanediol (EAB) triads. In the starting polyesters, only signals which could be assigned to the corresponding homopolymers (butanediol adipate: 24.33 ppm and ethylene glycol adipate at 24.17 ppm) were detected. The end product had the ratio of the triads BAB:(EAB+BAE):EAE=28:47:25 to be expected for a random copolymer.

### C. EXAMPLES OF THE COMBINED TRANSESTERIFICATION/GLYCOSYLATION OF POLYESTEROLS

Identical polyesterols derived from adipic acid and diethylene glycol (polydiethylene glycol adipate) and from adipic acid and 1,4-butanediol (1,4-polybutanediol adipate) were used in all the following examples of the transesterification and glycosylation of polyesterols in each case. The polydiethylene glycol adipate had a mean molecular weight of 2600 g/mol, a base number (hereinafter referred to as “OHN”) of 43 mg KOH/g and an acid number (hereinafter referred to as “AN”) of 0.8 mg KOH/g. The polybutanediol adipate had a mean molecular weight of 2350 g/mol, a base number of 45 mg KOH/g and an acid number of 0.7 mg KOH/g.

The polydiethylene glycol adipates and the polybutanediol adipates were each prepared as follows for all the following examples and comparative examples of the transesterification of polyesterols (process step a)):

Preparation of Polybutanediol Adipate:

39.3 kg of 1,4-butanediol were placed in a 250 l stirred tank reactor provided with a column and a stirrer. At 90° C., 57.3 kg of adipic acid were added via a pot. The reaction mixture was heated at 40° C./h to 240° C. The water of reaction formed was removed from the reactor by distillation. After a reaction time of 3 hours, the reactor pressure was reduced from atmospheric pressure to 30-50 mbar. After a reaction time of 24 hours, the acid number of the polyesterol prepared according to step a) was 0.6 mg KOH/g, the OH number was 45 mg KOH/g, and the water content immediately after the end of the reaction was <0.01% by weight.

Preparation of Polydiethylene Glycol Adipate:

57.7 kg of diethylene glycol were placed in a 250 l stirred tank reactor provided with a column and a stirrer. At 90° C., 73.0 kg of adipic acid were added via a pot. The reaction mixture was heated at 40° C./h to 240° C. The water of reaction formed was removed from the reactor by distillation. After a reaction time of 3 hours, the reactor pressure was reduced from atmospheric pressure to 30-50 mbar. After a reaction time of 24 hours, the acid number of the polyesterol prepared according to step a) was 0.8 mg KOH/g, the OH number was 43 mg KOH/g, and the water content immediately after the end of the reaction was <0.01% by weight.

### C1: Transesterification and Glycosylation of Polyesterols using 10% of Novozym 435 at 70° C.

120 g of a diethylene glycol adipate (OHN=43 mg KOH g, AN=0.8 mg KOH/g) and 123 g of a 1,4-butanediol adipate (OHN=45 mg KOH g, AN=0.6 mg KOH g) were mixed by stirring in a four-necked flask provided with a stirrer, reflux condenser and nitrogen inlet. The mixture was heated to 70° C. and evacuated for 4 hours. After admission of nitrogen, 25 g of dried Novozym 435 and 5 g of ethylene glycol and 5 g of 1,4-butanediol were added to the reaction mixture. The viscosity of the mixture was 850 mPas at 75° C.

The drying of the Novozym 435 was effected by storage of the enzyme at 70° C. and a pressure of 1 mbar for 12 hours in a vacuum drying oven.

The reaction was continued at 70° C. for 18 hours. The end product had an acid number of 0.4 mg KOH/g, an OH number of 99 mg KOH/g and a water content of 0.04% by weight. The viscosity was 200 mPas at 75° C.

The decrease in the viscosity from 850 mPas to 200 mPas is an index of the reduction in the mean molecular weight of the base polyesterols and thus of the incorporation of the diols into the polyesterol chains.

### C2: Transesterification and Glycosylation of Polyesterols using 10% of Novozym 435 at 70° C.

123 g of a diethylene glycol adipate (OHN=43 mg KOH/g, AN=0.8 mg KOH/g) and 123 g of a 1,4-butanediol adipate (OHN=45 mg KOH/g, AN=0.6 mg KOH/g) were mixed by stirring in a four-necked flask provided with a stirrer, reflux condenser and nitrogen inlet. The mixture was heated to 70° C. and evacuated for 4 hours. After admission of nitrogen, 25 g of dried Novozym 435 and 5 g of ethylene glycol were added to the reaction mixture. The viscosity of the mixture was 950 mPas at 75° C.

The drying of the Novozym 435 was effected by storage of the enzyme at 70° C. and a pressure of 1 mbar for 12 hours in a vacuum drying oven.

The reaction was continued at 70° C. for 18 hours. The end product had an acid number of 0.4 mg KOH/g, an OH number of 78 mg KOH/g and a water content of 0.03% by weight. The viscosity was 350 mPas at 75° C. after the reaction was complete.

The decrease in the viscosity from 950 mPas to 350 mPas is an index of the reduction in the mean molecular weight of the base polyesterols and thus of the incorporation of the diols into the polyesterol chains.

### D1: Comparative Example of the Enzymatic Transesterification of Polyesterols Having a High Molecular Weight at a Total Water Content of 0.5% by Weight (cf. Kumar et al.)

50 g of a diethylene glycol adipate (as in example C) and 50 g of an ethylene glycol adipate (OHN=56 mg KOH/g, AN=0.6 mg KOH/g) were mixed by stirring in a four-necked flask provided with a stirrer, reflux condenser and nitrogen inlet. The mixture was heated to 70° C. and evacuated for 4 hours. After admission of nitrogen, 10 g of dried Novozym 435 and 0.8 g of water were added to the reaction mixture. The water content after the addition of water was 0.8% by weight.

The drying of the Novozym 435 was effected by storage of the enzyme at 70° C. and a pressure of 1 mbar for 12 hours in a vacuum drying oven.

The reaction was continued at 70° C. for 10 hours. The end product had an acid number of 45 mg KOH/g, an OH number of 100 mg KOH/g and a water content of 0.5% by weight. The viscosity was 150 mPas at 75° C.

This comparative experiment shows that a water content of only 0.5% by weight leads to polyesterols having a high acid number and that a total water content of 0.8% by weight during the enzymatic transesterification according to process step b) leads to polyesterols having high acid numbers.

### D2: Comparative Example of the Enzymatic Glycosylation of Polyesterols Having a Low Molecular Weight at a Total Water Content of 0.14% by Weight (cf. Kumar et al.)

98 g of an ethylene glycol adipate (OHN=56 mg KOH/g, AN=0.6 mg KOH/g) were mixed by stirring in a four-necked flask provided with a stirrer, reflux condenser and nitrogen inlet. The mixture was heated to 70° C. and evacuated for 4 hours. After admission of nitrogen, 10 g of dried Novozym 435 and 2 g of 1,6-hexanediol were added to the reaction mixture. The water content after the addition of 1,6-hexanediol was 0.15% by weight.

The drying of the Novozym 435 was effected by storage of the enzyme at 70° C. and a pressure of 1 mbar for 12 hours in a vacuum drying oven.

The reaction was continued at 70° C. for 10 hours. The end product had an acid number of 10 mg KOH/g, an OH number of 78 mg KOH g and a water content of 0.14% by weight. The viscosity was 150 mPas at 75° C.

This comparative experiment shows that a water content of only 0.14% by weight leads to polyesterols having a high acid number and that a total water content of 0.15% by weight during the enzymatic glycosylation according to process step b) leads to polyesterols having high acid numbers.

1-12. (canceled)

13. A two-stage process for preparing polyesterols, which comprises the following process steps:
a) preparation of at least one base polyesterol by reaction of in each case at least one dicarboxylic acid with in each case at least one polyhydroxyl compound,
b) reaction of the base polyesterol from a) or a mixture of the base polyesterols from a) with at least one enzyme and, if appropriate, with further polyhydroxyl compounds, wherein the base polyesterols, the enzymes and, if appropriate the further polyhydroxyl compounds together have a water content of less than 0.1% by weight.

14. The process according to claim 13, wherein the reaction according to step b) is carried out without solvent.

15. The process according to claim 13, wherein base polyesterols, enzymes and, if appropriate, further polyhydroxyl compounds which together have a water content of less than 0.05% by weight are used in process step b).

16. The process according to claim 13, wherein the at least one base polyesterol from process step a) is prepared under an inert gas atmosphere.

17. The process according to claim 13, wherein the at least one base polyesterol from process step a) is temporarily stored under an inert gas atmosphere prior to the reaction according to process step b).

18. The process according to claim 13, wherein the at least one base polyesterol from process step a) is dried prior to the reaction according to process step b).

19. The process according to claim 13, wherein the reaction according to step b) is carried out at 20 110° C.

20. The process according to claim 13, wherein the at least one enzyme is a lipase or hydrolase.

21. The process according to claim 13, wherein the at least one enzyme is a lipase and is selected from among the lipase Candida antartica and the lipase Burkholderia plantarii.

22. The process according to claim 13, wherein the at least one enzyme is used which is immobilized on a support material.

23. A polyesterol obtainable by a process according to claim 13.

24. The polyesterol according to claim 23 which has an acid number of less than 3 mg of potassium hydroxide per gram of polyesterol.
